(19) United States
(12) Patent Application Publication
(10) Pub. No.: US 2026/0011974 A1
(43) Pub. Date: Jan. 8, 2026

(54) Semiconductor Laser Assemblies for Medical Applications

(71) Applicant: SemiNex Corporation, Danvers, MA (US)

(72) Inventors: Ronald Moore, Boxford, MA (US); Matthew Hamerstrom, Salem, MA (US); Dennis Donahue, Wilmington, MA (US)

(21) Appl. No.: 19/237,176

(22) Filed: Jun. 13, 2025

Related U.S. Application Data
(60) Provisional application No. 63/666,826, filed on Jul. 2, 2024.

(51) Int. Cl. H01S 5/024; H01S 5/0683; A61B 18/00; A61B 18/20

(52) U.S. Cl. CPC H01S 5/024; H01S 5/06832; A61B 2018/00476; A61B 18/203

(57) ABSTRACT

Medical applications for the devices disclosed in this patent include reducing wrinkles, acne, scar tissue, warts, and brown spots, as well as cauterizing wounds, promoting wound healing and relieving chronic joint pain in knees, shoulders, elbow, and hands. The devices disclosed in this patent all use the same semiconductor device where the device is packaged to create low cost, compact, portable and handheld medical devices for the applications described herein.

### RELATED APPLICATIONS

This application claims the benefit under 35 USC 119(e) of U.S. Provisional Application No. 63/666,826, filed on July 2, 2024, which is incorporated herein by reference in its entirety.

### BACKGROUND OF THE INVENTION

Semiconductor laser assemblies are commonly used in medical applications such as wrinkle reduction, scar and acne treatment, and relief of joint pain. In typical systems, a semiconductor laser diode is mounted in a metal submount and press-fitted into a heat sink for thermal management. One such system is shown in FIGS. 1–4 (Prior Art), where the laser diode is mounted in a modified TO-9 can press-fitted into a cylindrical heat sink bore and sealed with an optical lens. These prior designs often require tight tolerances for assembly, which can result in distortion of the laser mount, reduced thermal performance, and low manufacturing yield. These limitations increase cost and reduce reliability, especially for low-cost, handheld medical devices intended for home or personal use.

### SUMMARY OF THE INVENTION

The present invention provides improved semiconductor laser assemblies for medical applications, featuring stress-relieved heatsinks and precision mounting features to enhance manufacturability and reliability. In some embodiments, slots or internal broaches are incorporated into the heat sink to allow flexure during press-fit assembly, reducing mechanical stress on the laser mount. In other embodiments, the laser mount includes alignment features such as dowel pin slots to ensure proper orientation with respect to the heatsink and optical lens. The assemblies support various optical configurations, including cylindrical lenses for beam shaping, and are operable at different wavelengths suited for skin treatment or hair removal. These improvements enable the manufacture of compact, portable, and cost-effective laser-based medical devices.

In general, according to one aspect, the invention features a laser assembly with a stress relieved finned heatsink with no internal structures for alignment during manufacturing, with a press fitted laser diode and a lens attached at the exit of the heatsink to form a seal between the laser diode and the outside.

In embodiments, the finned heatsink is slotted with a slot that is 2mm, 1mm, 0.5mm or smaller through the inner shell of the heatsink to enable it to flex. The finned heatsink is broached in 1, 2, 3 or more places with a heatsink wall thickness of less than 1mm, 0.5mm or smaller which enables it to flex. The finned heatsink is copper and its alloys or aluminum and its alloys.

The laser diodes are mounted on a copper submount with slots for dowel pins for precisely orientating the laser diodes with respect to the heatsink and the output lens during the pressing operation, or the laser diodes are press fitted into the inner bore of the heatsink to a depth determined by the fixture which is the spacing required for the lens to properly expand the output, among other examples.

In various embodiments, to circularize the divergence, the laser assembly uses a lens, a bi-convex cylindrical lens, or a bi-concave cylindrical lens.

The laser assembly uses a window to illuminate a pre-determined treatment area, in one example.

A heatsink slot of the finned heatsink is sealed with a silicon sealant, a low outgassing epoxy, or a thermoplastic. In another example, the heatsink slot is laser welded after assembly. In some embodiments, an insert made up of copper or aluminum and their alloys is used to fill the heat sink slot, which is sealed with a silicon sealant or a low outgassing epoxy or is laser welded into place after assembly.

In some embodiments, the press fitted laser diodes are sealed on the back side with silicon, a low outgassing epoxy, an elastomer plug with two holes for the leads, or a rubber plug with two holes for the leads.

The laser assembly is outfitted with one laser diode that can operate at any one of the wavelengths in the range of 1300nm-1600nm for treatment of wrinkles, scars, brown spots and acne, outfitted with one laser diode that can operate at any one of the wavelengths in the range of 800nm-1100nm for hair removal and the treatment of wrinkles, scars, brown spots and acne, outfitted with any laser diode that can operate within the wavelength range of 800nm to 1600nm, or outfitted with a multi-junction laser diode operating at 790800808, up to 880nm to provide a larger treatment area for hair removal, to list a few examples.

In one embodiment, all clocking and alignment is accomplished with external fixtures.

The laser assembly uses pulse width modulation, pulse position modulation, power modulated pulses, variable pulse widths, variable pulse spacing, and/or variable power levels to control the temperature of the skin and hair follicles throughout the process.

The laser assembly is operated continuously while continuously varying the power to control the skin and hair follicles throughout the process, in one example.

The laser assembly can be used with a pyrometer to control the temperature of the skin with a feedback loop.

In one embodiment, the laser assembly is incorporated into a handheld device powered by an external power supply and/or a battery.

The power level of the laser assembly is controlled by a micro-controller.

In various embodiments, the laser assembly has a heat sink with any arbitrary fin design that can be symmetric or not symmetric, has a heat sink where the fins form circular cross section that matches the fan cross section to be used, or has a heat sink with a non-circular cross section that does not match the cross section of the fan, but can be easily mated to a fan or multiple fans where the number of fans is 1 or more.

In general, according to another aspect, the invention features a method of treating tissue using a laser assembly with a stress relieved finned heatsink with no internal structures for alignment during manufacturing, with a press fitted laser diode and a lens attached at the exit of the heatsink to form a seal between the laser diode and the outside. The method comprises positioning the laser assembly at a treatment site and operating the laser diode of the assembly to emit optical energy to the treatment site.

In general, according to another aspect, the invention features a method of assembling a laser assembly. The method comprises providing a stress relieved finned heatsink having no internal structures for alignment during manufacturing, press-fitting a laser diode into a bore of the heatsink, and attaching a lens or window at an output end of the heatsink to form a seal.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a side view of a prior art cylindrical laser assembly with a press-fitted TO-9 mount for skin treatment applications;

FIG. 2 is an exploded view of the prior art assembly showing the semiconductor laser, heatsink, and lens components;

FIG. 3 is a schematic diagram showing pulse parameters used to elevate and maintain skin temperature for wrinkle treatment;

FIG. 4 shows timing diagrams illustrating a sequence of laser pulses used to control skin heating;

FIG. 5 is a side view of a heatsink with a longitudinal slot and lead-in chamfers for stress relief and improved assembly;

FIG. 6 is a side section view of a heatsink with internal broached grooves for strain relief during press-fit assembly;

FIG. 7A is a perspective view of a laser diode mount with dowel pin slots for alignment and enhanced thermal contact;

FIG. 7B is a back side view of the laser diode mount;

FIG. 8 is a perspective view of a pressing fixture used to align and insert multiple laser mounts into heatsinks simultaneously;

FIG. 9 is a diagram showing a bi-concave cylindrical lens used to circularize the laser beam divergence;

FIG. 10 is a schematic of the system configured for hair removal with a shortened standoff and higher power laser diode;

FIG. 11 is a chart comparing electrical-to-optical conversion efficiency between 808 nm and 13xx–15xx nm laser diodes; and

FIG. 12 is a simulation or graph showing skin temperature increase over time using an 808 nm laser diode for hair removal.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term "and/or" includes any and all combinations of one or more of the associated listed items. Also, all conjunctions used are to be understood in the most inclusive sense possible. Thus, the word "or" should be understood as having the definition of a logical "or" rather than that of a logical "exclusive or" unless the context clearly necessitates otherwise. Further, the singular forms and the articles "a", "an" and "the" are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

It will be understood that although terms such as “first” and “second” are used herein to describe various elements, these elements should not be limited by these terms. These terms are only used to distinguish one element from another element. Thus, an element discussed below could be termed a second element, and similarly, a second element may be termed a first element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

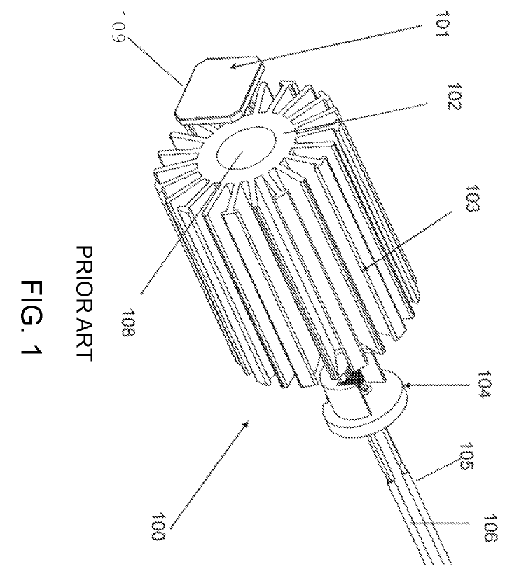

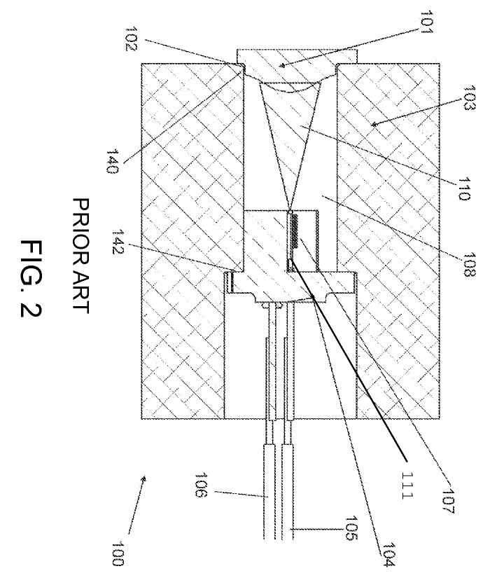

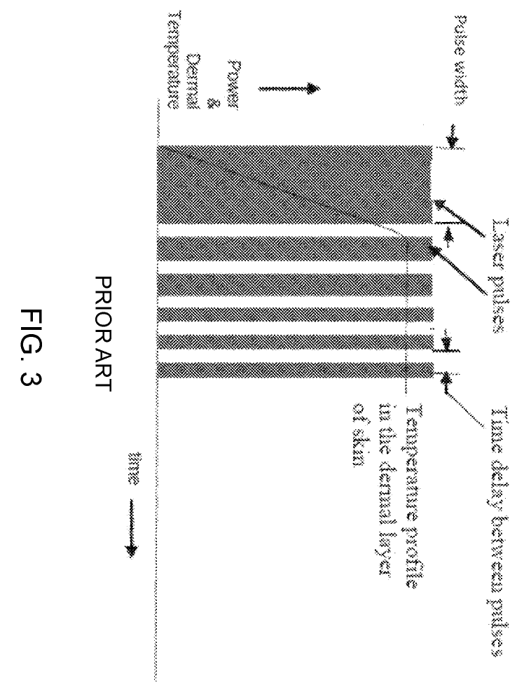

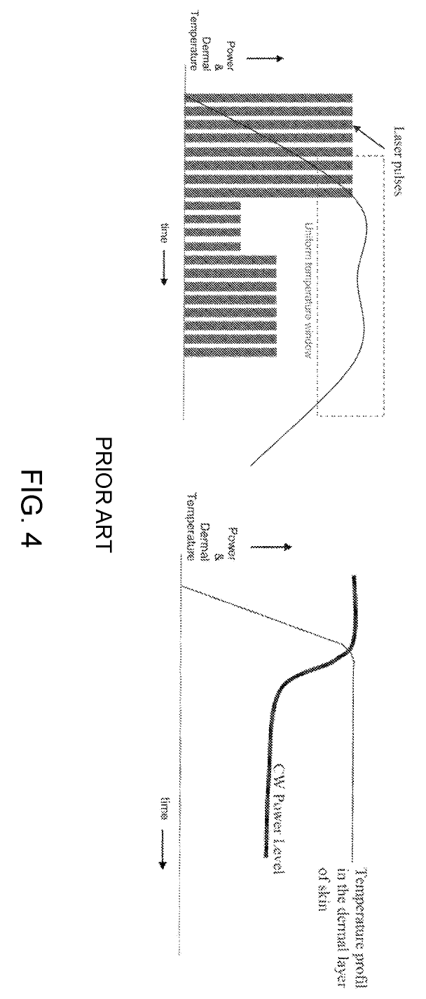

The device shown in FIG. 1 and 2 is used for stimulating collagen to reduce wrinkles using a range of pulse parameters (FIGS. 3 and 4) to elevate and maintain the skin temperature at or just below 45^{o}C. Above this temperature the person using the laser will experience discomfort and perhaps pain. The device consists of a single semiconductor laser (107) with a wavelength between 1380nm and 1570nm mounted on a modified TO-9 submount (104). This design is cylindrical symmetry with the laser mounted in the bore (108) of the cylindrical heat sink (102). The heat sink body (103) serves as the main housing or heat exchanger for the assembly, providing thermal mass and surface area for heat dissipation, and includes the bore (108) for receiving the TO-9 submount (104). A representative beam path (110) is shown within the bore (108), corresponding to the trajectory of light emitted from the laser diode (107) toward the output lens or window. The laser diode is mounted on a modified TO-9 submount (104) and either press fit into the bore up to a detent (142) or epoxied in place with thermal epoxy at the same location (142). This design approach results in a poor thermal performance for the laser diode, but just meets the requirements of the intended application of collagen stimulation. The end of the laser bore is sealed with a custom aspheric lens (101) or window depending on the spot required for the application. The lens (101) can be molded glass, polished glass or molded plastic lens with or without an anti-reflection coating, the current design uses lenses without the anti-reflection coating to reduce cost. The lens (101) is also positioned mechanically by the detent (140) in the opposite end of the bore (102) and is affixed in the bore with a low outgassing epoxy or a UV cure epoxy. The electrical power is delivered to the semiconductor device (107) mounted in the modified TO-9 can base (104) through two copper wires which are soldered to the leads (105, 106) exiting the package. The lower edge 109 of the lens 101 is preferably used for rotational clocking during assembly.

The device shown in FIGS. 1 and 2 has sufficient heat sink capacity to allow the semiconductor device (107) to operate at power levels up to 5 Watts pulsed or for short periods continuously. The ability to stimulate collagen requires the skin temperature to be between 39^{o}C and 45^{o}C. This can be accomplished as shown in FIG. 3, where a specific pulse duration followed by a varying pulse duration, pulse duty cycle is applied to first increase the temperature of the skin to the appropriate level followed by a lower duty cycle energy application to maintain the skin temperature for a period of 1 to 1.5 seconds. A specific example of a pulse sequence is shown in FIG. 4 where a first series of pulses increase the skin temperature, followed by a second lower power level series of pulses to reduce the rate of increase of the skin temperature and finally a third higher power level series of pulses to maintain the skin temperature for the pre-determined time. This same result can be obtained by using continuous laser power (CW) as shown in FIG. 4. In all these power control cases it is important to control the power density on the surface of the skin, this is accomplished with a mechanical standoff with a built-in contact sensor.

At low power levels, the same device can be used to treat joint pain. Low Level Light Therapy (LLLT) has been shown in numerous studies to be beneficial in reducing inflammation, promoting cellular regrowth and reducing pain.¹ The power densities are lower than with the first device described, and the treatment durations are typically performed at a wavelength between 830nm and 1060nm with a low power level (1 Watt) laser source. Again, the power density on the area of treatment must be maintained below the pain threshold as well as below the point at which burns may occur. This is again accomplished with a mechanical standoff mechanism with a contact sensor. The device if FIG. 1 can be configured to operate at these wavelengths to provide the LLLT treatment.

The invention described in this patent is several modifications to the original heatsink design (100) that greatly improve manufacturability, reliability and cost of the laser engine enabling more handheld laser medical applications. The original design can meet the exposure times described in FIGS. 3-4 for the wrinkle removal application as shown in patent number 11,135,444 B2. The heatsink size when combined with the appropriate fan enables the laser to operate for sufficient time for the treatment to be performed numerous times before the batteries need to be recharged. The markets targeted by the product are the low-cost home markets, which require the device cost to be low and the reliability to be on the order of a few hundred to 1000 hours. To meet the low-cost requirements, the manufacturing yield must be greater than 90%, which the original design outlined in patents 8,811,439 B2, 9,537,284 B2 and 10,686,293 B2, which are incorporated herein by this reference in their entirety, struggled to achieve. This instant patent application is about improvements to the design that increase the manufacturing yield substantially making manufacturing of the product economical and capable of meeting the operating requirements outlined in patent number 11,135,444 B2.

The key yield driver in the original design is the tight tolerances between the modified TO-9 can (104) and the bore (108). The modified TO-9 can (104) is manufactured out of copper and is press fitted into the bore (108) up to a detent (142) in the original patent. Because this is a press fit the manufacturing tolerances of the two parts, the modified TO-9 can (104) and the heatsink bore (108) are very tight which results in additional manufacturing costs and can affect yield if the parts are out of tolerance.

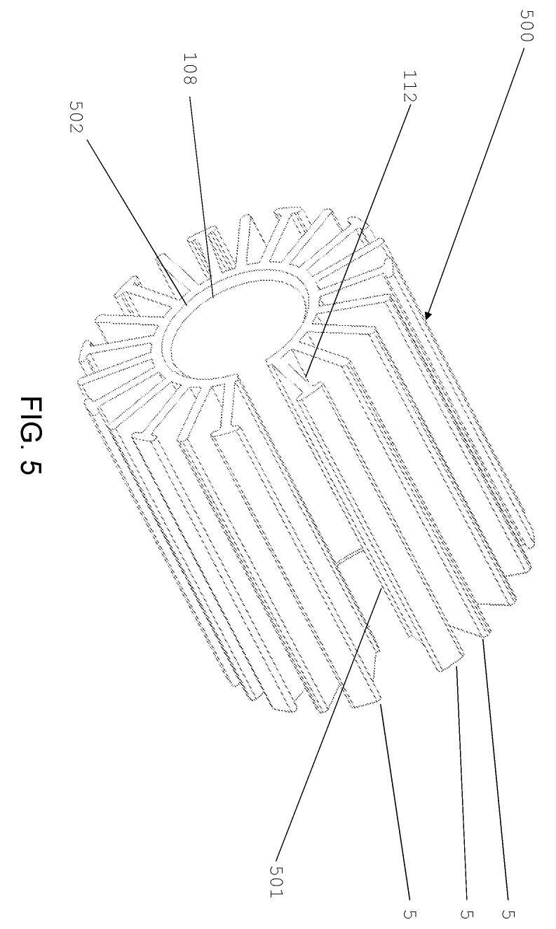

The first effort shown in FIG. 5 to improve the yield was the addition of lead-in chamfers (502) which assist in the alignment of the modified TO-9 can (104) to the heat sink (500) during assembly. While the alignment issues were minimized the yield failed to improve due to the tight tolerance on the outside diameter of the modified TO-9 can (104) and the bore in the heatsink (108).

Numerous assembly steps have been tried to improve the manufacturability including changing lead in chamfers on the bore, varying pressure and varying the speed of assembly when forcing the modified TO-9 can (104) into the bore (108) with no improvement in manufacturing yield. Since the modified TO-9 can mount (104) is manufactured from copper, it is easily distorted if the tolerances between the bore (108) and the can (104) are too tight due to variations in the manufacturing process of the two components. Any distortion of the semiconductor (107) mounting surface (111) will cause the device to fail.

The solution to relieving the tension on the semiconductor mounting surface (111) is to relieve the pressure exerted by the bore (108) on the modified TO-9 can mount (104). FIG. 5 shows a modification of the heatsink (500) with a slot (501) milled the entire length of the heat sink. The slot (501) allows the heatsink bore to spread slightly when the modified TO-9 can mount (104) is forced into the bore (108). This reduces the deformation of the semiconductor mounting surface (111) of the modified TO-9 can mount (104). A second modification which greatly aids in the assembly of the laser engine is the addition of lead-in chamfers (502) on both sides of the heat sink. A third modification is the elimination of the detents (140, 142) allowing the bore to be continuous throughout the part. The heatsink is now symmetric with no specific orientation which reduces the handling time by eliminating the need to inspect each part for its orientation. The heatsink (500) includes a back face (112) that provides structural support and may serve as a contact surface for mounting or additional thermal dissipation.

The width of the slot (501) milled into the heat sink can be 2 mm, 1 mm, 0.5 mm or any suitable break in the heat sink that relieves the constraint of the bore and allows the heatsink to flex slightly when the modified TO-9 can mount (104) is press into the bore. Adding this stress relief to the heatsink greatly improved the yield of the manufacturing process by reducing the deformation of the modified TO-9 can mount (104) and therefore the deformation of the semiconductor mounting surface (111). The final product must be sealed from the environment to prevent dust and humidity from entering the interstitial area between the modified laser diode heatsink (104) and the output lens. The slot may be filled with a low outgassing glue, epoxy or silicon. In another embodiment, a metal plate or heatsink fin may be bonded in place with a low outgassing glue or epoxy that improves the thermal performance of the heatsink without impacting the stress on the semiconductor mount (111). The slot may be located between any two of the fins on the heatsink without disrupting the fin layout. The preferred embodiment is located between the two fins with tabs on their ends (503, 504) just to the side of the three identical fins (502) with this position eliminating a single fin from the heatsink.

The lead in chamfers (502) on both sides of the heat sink are designed to provide a smooth self-alignment of the bore to the modified TO-9 can (104) regardless of which direction the parts are assembled. The lead-in chamfers are designed to be at a shallow angle of 60°, 70° or 80° with respect to the face of the heatsink and are located on both ends of the heatsink bore (108).

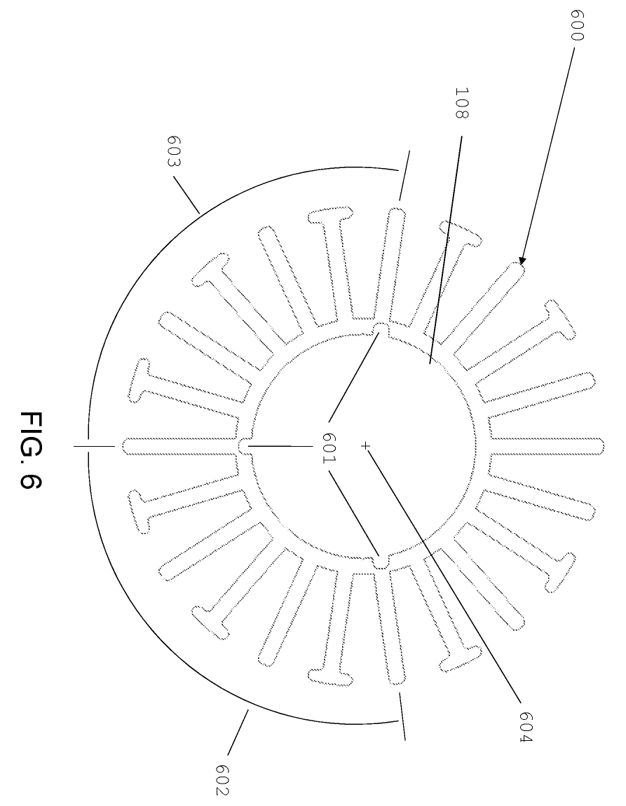

A second embodiment of the laser engine heatsink is shown in FIG. 6, here the stress relief is provided by broaching 1, 2, 3 or more grooves on the inner diameter of the heat sink. These grooves leave a wall thickness of 1mm, 0.5 mm, 0.2 mm or less that provides the necessary strain relief when the modified TO-9 can (104) is press fit into the bore (108). The grooves may be a variety of shapes, rectangular, semi-circular, or triangular and they may be located under fins as depicted in FIG. 6, or not under fins. They may be separated by an equal angle (602, 603) or an uneven angle (602, 603), the preferred embodiment is an even angular spacing. The grooves may be aligned with the center (604) of the heat sink, or they may not, the preferred embodiment is that they are aligned to simplify manufacturing.

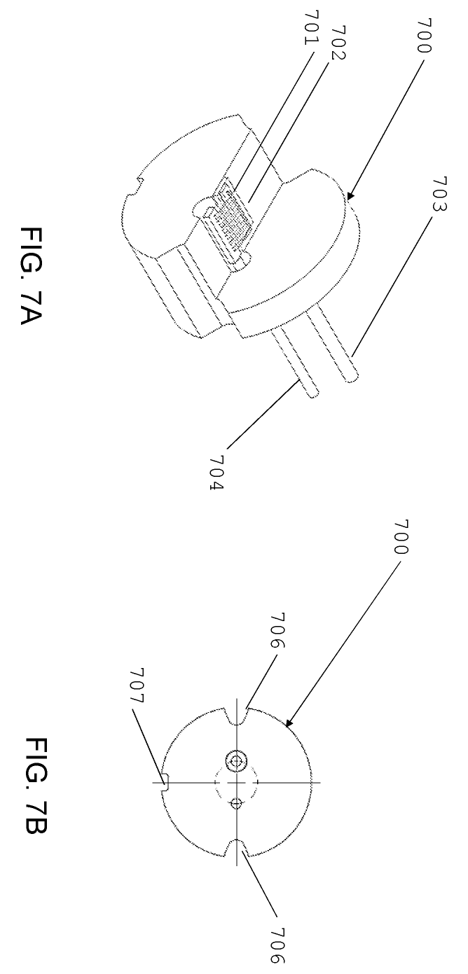

The third embodiment of the laser engine modification is shown in FIGS. 7A (perspective view) and 7B (back side view), where the semiconductor mount (700) improves the thermal performance, the clocking of the parts and the positioning of the parts. The mount (700) is designed to provide a greater contact area between the mount (700) and the bore (108) of the heatsink than the previously modified TO-9 can design. Other features of the mount (700) are the slots (706) located on either side of the mount (700) that are designed to accept dowl pins (806) to provide clocking of the assembly (700) relative to the heatsink (500, 600) which has a separate but properly orientated fixture to hold the heatsink (500, 600) in a pre-determined position. These slots (706) are larger than what is typically used with a TO-9 mount and are shaped so when the dowel pins (806) are seated in the mount (700), the mount is precisely located with respect to the heatsink (500, 600). The mount (700) further includes one or more axial indentations or relief channels (707), which extend along its outer surface to reduce insertion stress, improve mechanical compliance, or assist in alignment during press-fit assembly. This is an important feature when aligning the output optic which is cylindrical symmetry. The semiconductor laser diode (701) is mounted on a submount (702) which is affixed to the heatsink (700). Electrical contacts include a pin (703) fused to the back of the mount to provide the ground contact and a separate isolated pin (704) to provide the p-contact or n-contact bias. The preferred embodiment is for the device to be mounted p-side down with the n-side contact biased by a negative voltage.

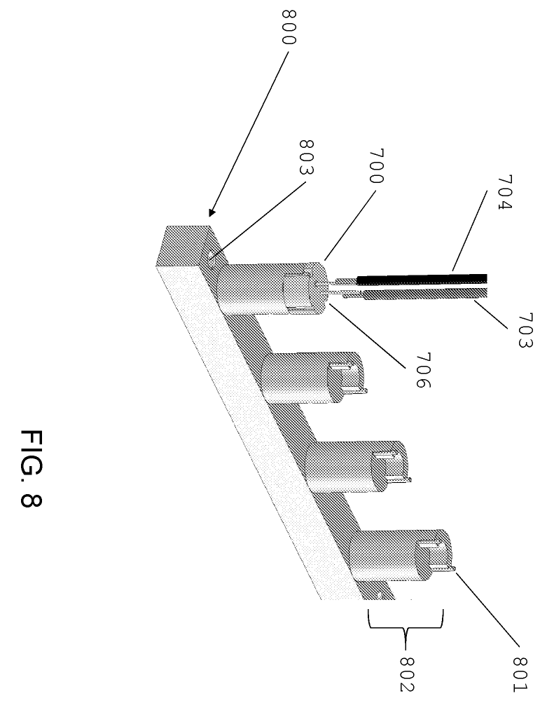

FIG. 8 is a fourth embodiment of the laser engine modifications that allows for the detents (140, 142) to be removed from the heatsink, lowering its manufacturing costs. This is accomplished with the pressing fixture (800) which is used to press the laser diode assemblies (700) into the heat sink (500, 600). The fixture incorporates dowel pins (801) which are captured by the dowel pin slots (706) on the semiconductor mounts (700). The fixture may also include one or more seating recesses or bore holes (803) formed in the surface of the base, which are positioned to align with corresponding features on the mounts (700) to aid in registration or mechanical stability during pressing. As the laser diode assemblies (700) are pressed into the heat sink, the laser diode assembly positions are set by the height (802) of the pressing fixture (800). This fixture (800) can press up to 4 assemblies simultaneously, however it can be designed to press 1, 2, 3, 4, 10, 20 or more assemblies at once. With the heat sink relieved of the stress on the laser diode assembly (700) by the slot (501) or broaches (601) the amount of pressure that must be applied to press the laser diode assemblies into the heatsinks (500, 600) is greatly reduced. This innovation enables a larger number of assemblies to be pressed simultaneously.

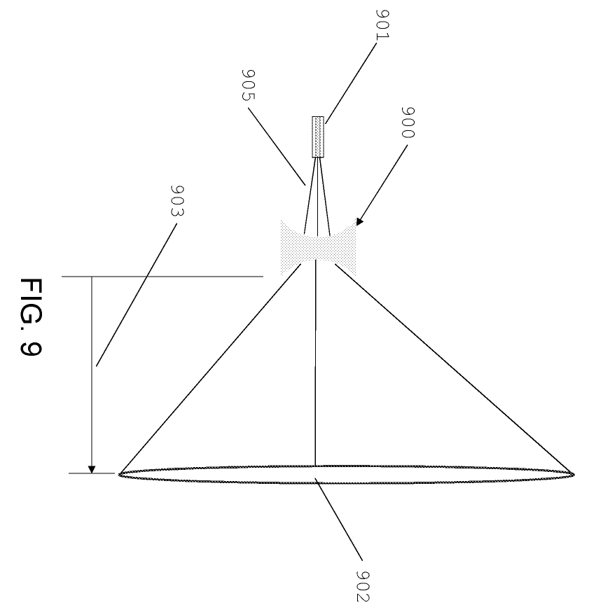

As shown in FIG. 9, a bi-concave lens (900) is used to expand the slow axis of the laser, which is typically 10°, 11°, 12°, 13° FWHM or more to the same divergence as the fast axis, which is typically 30°, 40°, 50° FWHM or more depending on the transverse waveguide design. The laser beam originates from a semiconductor laser diode (901) and enters the bi-concave lens (900), which expands the divergence in the slow axis before the beam reaches the treatment plane (902). The preferred embodiment is 13° FWHM (905) for the slow axis and 30° FWHM for the fast axis. The bi-concave lens (900) increases the slow axis from 13° to 30° resulting in a spot that is approximately circular at the working distance of 21mm. This working distance (903) is the distance between the output of the lens (900) and the treatment plane (902), and defines the point at which the beam achieves the desired optical profile. The beam is approximately 19.5 mm x 17.5 mm at the treatment plane (902) with an average power density of ~150 mW/cm². The power density can be less than 150 mW/cm² or more than 150 mW/cm² but the energy requirements for the process remain the same so the pulse parameters shown in FIGS. 3, 4 and 5 must be adjusted to maintain a skin temperature below 45 °C. This system design is suitable for wrinkle removal as well as treatment of other skin issues such as acne and brown spots.

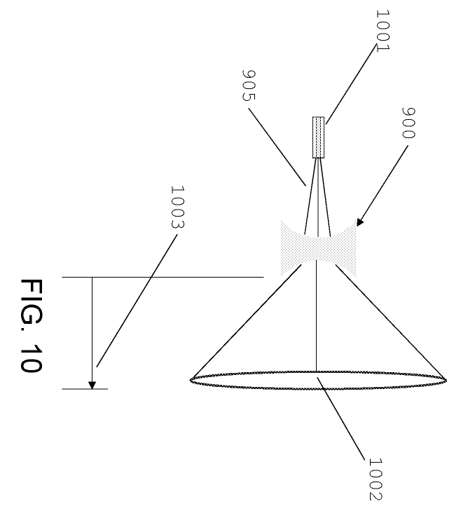

As shown in FIG. 10, this same system can be reconfigured with a different laser diode (1001) that operates at 808 nm to 1064 nm to address the hair removal market. This application requires a higher power density than the previous application, so the system is operated at a shorter standoff distance of 10 mm (1003) to create a beam that is approximately 10 mm x 10 mm (1002). The same heatsink (500, 600), semiconductor mount (700) assembly fixture method (800) and lens assembly (900) can be used in fabricating this device. The assembly fixture will have to be slightly modified (802) to accommodate the higher index of refraction of the lens which increases the power of the optic and shortens the virtual focal length (905).

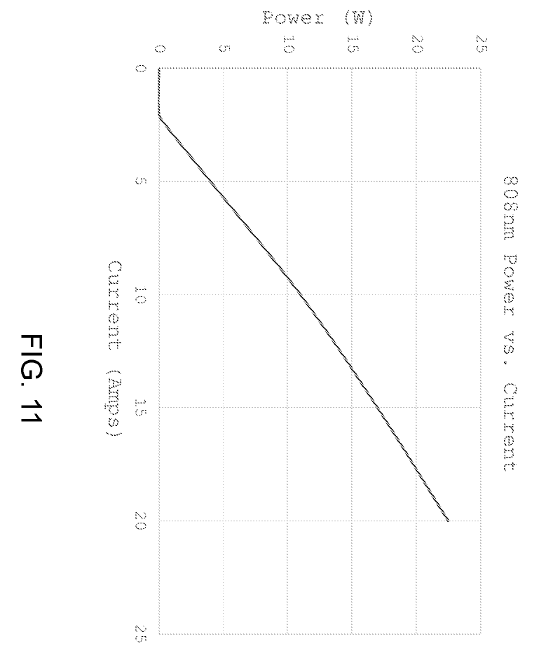

An 808 nm laser diode is substantially more efficient than a 13XX-15XXnm laser diode as shown in FIG. 11. This is a commercial laser diode that operates with an electrical to optical efficiency of 65% compared to a 13XX-15XX laser diode that has an electrical to optical efficiency of just 16%. This 4x improvement in efficiency means that a much higher output power laser diode can be used on the mount (700) with the same average heat load.

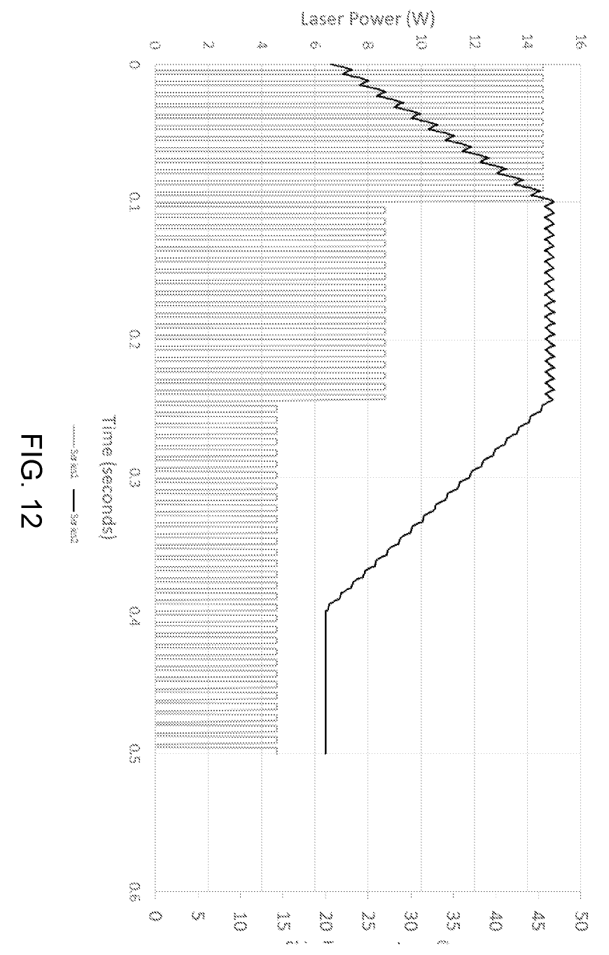

According to the literature, the 808nm laser diode wavelength has proven to be the most effective at hair removal, but it requires the follicle temperature to be raised above 45°C. As mentioned above, this is the pain threshold for many people, so the application requires that cooling be applied to the surface of the skin during the treatment. The analysis shown in FIG. 12 does not consider the cooling but shows that a single 808 nm laser diode with the power characteristics shown in FIG. 11 has sufficient power to increase the skin temperature to over 45°C and consequently the hair follicle. The literature indicates that hair removal can be accomplished with between 3 and 30 J/cm² energy deposition when the follicle temperature exceeds 45°C which this system can provide.

While this invention has been particularly shown and described with references to preferred embodiments thereof, it will be understood by those skilled in the art that various changes in form and details may be made therein without departing from the scope of the invention encompassed by the appended claims.

What is claimed is:

1. A laser assembly with a stress relieved finned heatsink with no internal structures for alignment during manufacturing, with a press fitted laser diode and a lens attached at the exit of the heatsink to form a seal between the laser diode and the outside.

2. The laser assembly of claim 1 where the finned heatsink is slotted with a slot that is 2mm, 1mm, 0.5mm or smaller through an inner shell of the heatsink to enable it to flex or is broached in 1, 2, 3 or more places with a heatsink wall thickness of less than 1mm, 0.5mm or smaller which enables it to flex.

3. The laser assembly of claim 1, wherein the finned heatsink is copper and its alloys or aluminum and its alloys.

4. The laser assembly of claim 1, wherein the laser diode are mounted on a copper submount with slots for dowel pins for precisely orientating the laser diode with respect to the heatsink and the lens during a pressing operation.

5. The laser assembly of claim 1, wherein the laser diode is press fitted into the inner bore of the heatsink to a depth determined by a fixture which is the spacing required for the lens to properly expand the output.

6. The laser assembly of claim 1, wherein the laser assembly uses a lens to circularize the divergence, which is a bi-convex cylindrical lens to circularize the divergence or a bi-concave cylindrical lens to circularize the divergence.

7. The laser assembly of claim 2, wherein the heatsink slot is sealed with a silicon sealant, a low outgassing epoxy, or a thermoplastic.

8. The laser assembly of claim 2, wherein the heatsink slot is laser welded after assembly or an insert made up of copper or aluminum and their alloys is used to fill the heatsink slot.

9. The laser assembly of claim 5, wherein the press fitted laser diodes are sealed on the back side with silicon, a low outgassing epoxy, an elastomer plug with two holes for the leads, or a rubber plug with two holes for the leads.

10. The laser assembly of claim 1, is outfitted with one laser diode that can operate at any one of the wavelengths in the range of 1300nm-1600nm for treatment of wrinkles, scars, brown spots and acne or can operate at any one of the wavelengths in the range of 800nm-1100nm for hair removal and the treatment of wrinkles, scars, brown spots and acne.

11. The laser assembly of claim 1, wherein the laser assembly is outfitted with a multi-junction laser diode operating at 790, 800, 808, or up to 880 nanometers to provide a larger treatment area for hair removal.

12. The laser assembly of claim 1, wherein the laser assembly uses pulse position modulation, power modulated pulses, variable pulse widths, variable pulse spacing, and/or variable power levels to control the temperature of the skin and hair follicles throughout the process.

13. The laser assembly of claim 1, wherein the laser assembly is operated continuously while continuously varying the power to control the skin and hair follicles throughout the process.

14. The laser assembly of claim 1, wherein the laser assembly is used with a pyrometer to control the temperature of the skin with a feedback loop.

15. The laser assembly of claim 1, wherein the laser assembly is incorporated into a handheld device powered by an external power supply or a battery.

16. The laser assembly of claim 1, wherein the power level of the laser assembly is controlled by a micro-controller.

17. The laser assembly of claim 1, wherein the heat sink has a non-circular cross section that does not match the cross section of the fan, but can be mated to a fan or multiple fans, wherein the number of fans is one or more.

18. A method of treating tissue using the laser assembly of claim 1, the method comprising:
positioning the laser assembly at a treatment site; and
operating the laser diode of the assembly to emit optical energy to the treatment site.

19. A method of assembling a laser assembly, the method comprising:
providing a stress relieved finned heatsink having no internal structures for alignment during manufacturing;
press-fitting a laser diode into a bore of the heatsink; and
attaching a lens or window at an output end of the heatsink to form a seal.
